SEQUENCE LISTING (3 sequences)

SEQ ID NO: 1 - DNA, 3207 bp, synthetic construct
  source 1..3207: mol_type = other DNA; note = nucleotide sequence of rhCol III a1
caatacgacagttatgatgtaaaaagtggagtggcagttggcggcctggctggttacccggggccagcgggaccaccgggcccgccgggtcccccgggtaccagcggccaccctggaagcccgggcagtccgggttaccagggtccgcccggtgaaccgggtcaagcaggtccgagcggccctcccggtccgccgggtgcaatcggccctagcgggccggctggtaaggacggggagagcggtcgccctggccgtccgggcgagcgcggcctgcccggccctccaggcatcaaaggtccggcgggcattccgggttttccgggtatgaaaggccatcgtggtttcgatggtcgcaatggtgagaaaggggagacgggtgcgcctggtctgaaaggcgaaaacggtctgccgggcgagaacggagcgccgggacctatgggtcctcgtggcgcgccaggtgaacgcggccgcccggggctgccgggggcggctggtgcgcgtggcaacgatggtgcgcgtggctccgatggccaacctggcccgcccggaccacccggcacggcaggttttccgggctcgccgggcgcgaaaggcgaagttggtccggcgggtagcccaggttctaatggcgcgccgggacaacgtggggagccgggtccgcagggccatgcaggggcgcagggcccgccaggaccgccggggatcaacggtagccctggcgggaagggcgagatgggtccggcagggattccgggagccccaggcctgatgggtgcccgcggacctcccggtccggctggggccaatggtgcgcctggactgcgtggtggtgctggcgaaccgggaaagaatggtgcgaaaggcgagccgggcccgcgcggcgaacgtggtgaagctggtatcccgggcgtgccgggtgctaaaggtgaagatggcaaggacgggtcacccggagaaccgggagcgaacggattacctggcgcggcgggagagaggggcgcgccagggtttcgtggcccagcgggtccaaatggaatcccaggcgagaaagggccggctggagaacgtggagcaccgggtccagcgggcccgcgtggtgctgcgggcgaaccggggcgtgacggtgtgccgggcggtccggggatgcgtggcatgcctggttcgccgggaggcccgggttccgatggtaaaccgggcccgccaggcagtcagggggagtccggacgtccgggcccgccgggaccatccggtcctcggggacaaccgggagtgatgggcttcccgggtccgaaaggaaacgatggggcccccggaaaaaacggcgagcgtggtggtccgggcggtccgggaccgcaaggacctccggggaagaacggtgaaaccggtccgcaaggcccaccgggccctacaggacctggaggcgataaaggtgacaccggcccgccgggtcctcaaggtcttcagggtctgccgggtacgggcgggccacctggcgagaatggtaagccgggtgaaccgggccccaagggtgacgccggtgcgccgggtgcacccggcggcaagggcgacgctggcgcaccgggcgaaagaggaccgccaggtttggcgggcgcgcccggactgcgtggcggtgccggtccaccgggtccggaaggtggcaaaggcgctgcggggccacccgggcctccgggcgcggcgggcactccgggtcttcaggggatgccgggcgagcgcggtggtttgggttccccgggtcccaagggtgacaaaggtgaaccgggcggcccaggtgcagacggcgttccgggtaaggacggtccacgtggcccgaccggtcctatcggccctcctggccctgcaggtcagccgggggataagggcgagggtggtgcgccaggactgccgggtatcgcaggtccgcgtggttccccgggcgagcgtggcgaaaccggcccaccgggacctgcgggttttccaggtgcgcctggacaaaacggggagccgggtggtaagggtgagcgtggcgcacctggtgaaaagggggaaggcggccctccgggtgttgcgggccccccggggggctcagggccagctggcccaccgggcccgcagggtgtgaaaggagagagaggttcgccgggaggtcccggtgccgccggtttccctggcgcgcgtggattgccgggaccgcctggtagcaatggtaacccgggcccgccgggaccgagcggttctccgggcaaggacggtcctccgggcccggccggtaacaccggtgcgcccgggtcgccgggtgtcagcggccccaagggcgatgcaggacaaccgggtgagaagggctctccgggtgcgcagggcccgccgggcgctccgggaccgctgggtattgctggaattaccggagcgcgtggactggccggtccgccgggtatgccggggccgcgcggcagcccagggccacagggtgtgaaaggcgagtctggaaagccgggtgcgaacgggctgagcggtgagcgcgggccaccaggcccgcagggtttgccaggccttgccggcaccgcgggagaaccggggcgtgatggaaatccgggttctgatggtctcccgggccgtgatggttccccggggggtaagggcgaccgcggtgagaacggtagcccgggtgcgccaggggcgccgggtcaccctggcccgccgggtcccgtcggtccggcgggcaaaagcggtgaccgtggtgagagcggtccggcggggccggccggcgcgcctggtccggcaggctctcgtggtgcgccaggtcctcaaggccccagaggcgacaagggcgaaaccggagaacgcggtgctgctggcattaaaggtcatcgtggcttcccgggtaacccaggcgcgccaggaagcccgggtccggcagggcagcagggtgccatcggttctccgggcccggcgggtccgcgagggccggttggcccgagcggtccgccgggtaaagacggcaccagcggtcacccgggccccatcggccccccgggtccgcgcggtaatcgtggcgaacgcggatcggaaggttccccgggtcacccaggtcagcctgggccgccgggtccgccgggggctccgggtccgtgttgcggtggcgttggggctgcagcgattgcagggattggtggggagaaagcaggcggtttcgccccttattatggttaa

SEQ ID NO: 2 - DNA, 819 bp, synthetic construct
  source 1..819: mol_type = other DNA; note = DNA of the TEV protease digestion sequence
atgagactgctgaccgccctgttcgcctacttcatcgtggccctgatcctggccttcagcgtgtccgccaagagcatgcaccaccaccatcaccatcatcacatgagcaagggcgaggaactgttcaccggcgtggtgcccatcctggtggaactggacggcgacgtgaacggccacaagttctctgtgcggggcgagggcgagggggacgccacaaatggcaagctgaccctgaagttcatctgcaccaccggaaagctgcccgtgccctggcctaccctggtcacaaccctgacctacggcgtgcagtgcttcagcagataccccgaccacatgaagcggcacgatttcttcaagagcgccatgcccgagggctacgtgcaagaacggaccatcagcttcaaggacgacggcacctacaagaccagagccgaagtgaagttcgagggcgacaccctggtcaaccggatcgagctgaagggcatcgacttcaaagaggacggcaacatcctgggccacaagctggaatacaacttcaacagccacaacgtgtacatcaccgccgacaagcagaagaacggcatcaaggccaacttcaagatccggcacaacgtggaagatggcagcgtgcagctggccgaccactaccagcagaacacccccatcggcgacggccccgtgctgctgcccgacaatcactacctgagcacccagagcgtgctgagcaaggaccccaacgagaagcgggaccacatggtgctgctggaatttgtgaccgccgctggcatcacccacggcgagaacctgtacttccaaggg

SEQ ID NO: 3 - protein, 1320 aa, synthetic construct
  source 1..1320: note = amino acid sequence of the rhCol III a1 fusion protein
KSMHHHHHHHHMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGENLYFQGGSQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYG

(19) United States
(12) Patent Application Publication
(10) Pub. No.: US 2025/0257119 A1
(43) Pub. Date: Aug. 14, 2025

(54) Recombinant Humanized Collagen Type III Alpha-1, and Expression Vector and Use Thereof

(71) Applicant: Shandong D-Nutrimec Biomedical Co., Ltd., Heze (CN)

(72) Inventors: Zhidong Guo, Heze (CN); Jiajia Shao, Heze (CN); Hongwei Ma, Heze (CN)

(21) Appl. No.: 18/834,531

(22) PCT Filed: Dec. 26, 2023
(86) PCT No.: PCT/CN2023/141727

§ 371 Date: Jul. 30, 2024

(30) Foreign Application Priority Data
Apr. 10, 2023 (CN) 202310368832.8

(51) Int. Cl. C07K 14/78; A61K 38/00; C12M 1/00; C12N 5/00; C12N 5/071; C12N 15/85

(52) U.S. Cl. CPC C07K 14/78; C12M 21/02; C12N 5/0037; C12N 5/0686; C12N 15/85; A61K 38/00; C07K 2319/02; C07K 2319/50; C07K 2319/60; C12N 2500/02; C12N 2523/00; C12N 2527/00; C12N 2800/22

(57) ABSTRACT

A recombinant humanized collagen type III alpha-1 (rhCol III a1), and an expression vector and use thereof are provided, relating to the technical field of bioengineering. A sequence on positions 154 to 1,221 of a human type III collagen α chain is ligated to an optimized vector pcDNA3.1, a resulting optimized expression vector is transfected into a human embryonic kidney cell Expi293F, and then rhCol III a1 is secreted to form a fusion protein with a soluble green fluorescent protein (GFP). The expressed rhCol III a1 that forms the fusion protein with the GFP shows an activity of promoting the migration of BALB/c 3T3 cells, thus exhibiting a great application potential in the field of skin care.

### TECHNICAL FIELD

The present disclosure belongs to the technical field of bioengineering and specifically relates to a recombinant humanized collagen type III alpha-1 (rhCol III a1), and an expression vector and use thereof.

### BACKGROUND

Collagen, as one of the most abundant proteins in mammals, accounts for 25% to 30% of total protein mass and is the main structural protein in the extracellular matrix found in various human connective tissues. Collagen participates in the composition of many human organ tissues, such as skin, cornea, neural retina, bones, and muscles. Collagen type III is one of the most abundant fibrillar collagens as well as a homotrimer formed by three α1(III) chains (Col3a1). Collagen type III in the skin gradually decreases with age, with a proportion from the fetal stage to the adult stage decreasing by 8% to 11%. Since being secreted by fibroblasts and other mesenchymal cell types, collagen type III becomes a major player in various inflammation-related pathologies. Collagen type III is involved in lung injury: viral and non-viral liver disease, renal fibrosis, hernia, and vascular diseases. Collagen type III and collagen type I are the major components of the interstitial matrix, and mutations in the collagen type III are associated with congenital connective tissue hypoplasia syndrome (also known as Ehlers-Danlos syndrome), vascular defects, and aortic aneurysms and aneurysms. In addition to its wide application in the field of skin care, collagen type III also has many exploratory studies that are expected to expand its cutting-edge applications. For example, biosynthetic corneas made from humanized collagen type III are optically clear and can replace donor therapy or genetically modified pig treatments without the need for immunosuppressive drugs. The humanized collagen type III can complete heart valve artificial synthesis and endometrial perfusion repair. Adding collagen type III to a tumor microenvironment can promote tumor cells to enter and maintain a dormant state, thus inhibiting tumor proliferation. Therefore, collagen type III shows a huge potential application market.

The collagen currently used in industry is mainly extracted from the skin or bones of pigs and cattle through acid, alkali, or enzymatic methods, or from the skin of deep-sea fish. Although the technology for extracting collagen from animal tissues is mature, there are problems such as immunogenicity and limited sources, making it difficult to meet the huge market demand. With the large-scale application of genetic engineering, the generation of recombinant collagen through genetic engineering has become the most promising method to solve the problem of limited collagen sources. Compared with animal-derived collagen, recombinant collagen has a short production cycle and low cost, is suitable for large-scale production, and can avoid problems such as animal protein immunogenicity and animal-derived diseases. Therefore, the preparation of recombinant collagen, especially recombinant humanized collagen, is currently a hot topic in collagen production research.

Full-length collagen has a large molecular weight (not less than 100 kDa) and exhibits a variety of protein post-translational modifications, such as proline and lysine hydroxylation as well as glycosylation. Currently, the domestic production of recombinant collagen type III uses an Escherichia coli expression system or a yeast expression system, and both of the expression systems have the advantage of large-scale expression. However, collagen obtained by the Escherichia coli expression system is mostly truncated, fragmented, or artificially modified based on the collagen sequence characteristics, and is quite different from the real collagen type III in the human body. Meanwhile, since protein post-translational modification in yeast is different from that in the human body, recombinant collagen obtained by the yeast expression system is quite different from humanized collagen.

### SUMMARY

In view of this, an objective of the present disclosure is to provide rhCol III a1, where the rhCol III a1 is fused with a green fluorescent protein (GFP) and has a desirable activity in promoting cell migration and a great application potential.

The objective of the present disclosure is also to provide an expression vector of the rhCol III a1, where sequence on positions 154 to 1.221 of a humanized collagen α chain is ligated to an optimized vector pcDNA3.1 to construct the expression vector capable of expressing the rhCol III a1.

The objective of the present disclosure is also to provide an expression method of the rhCol III a1, where an rhCol III a1 chain is expressed in a human embryonic kidney cell Expi293F, and a fusion expression plasmid is used to increase a protein expression level to realize the expression of a full-length segment of the rhCol III a1.

To achieve the above objective, the present disclosure provides the following technical solutions.

The present disclosure provides an rhCol III a1, where rhCol III a1 has the nucleotide sequence set forth in SEQ ID NO: 1.

The present disclosure further provides an expression vector for rhCol III a1, including the nucleotide sequence set forth in SEQ ID NO: 1, a gene sequence of a secretion signal peptide, a gene sequence of a GFP, and a DNA of a tobacco etch virus (TEV) protease digestion sequence.

Preferably, a preparation method of the expression vector includes the following steps: subjecting the nucleotide sequence set forth in SEQ ID NO: 1, the gene sequence of the secretion signal peptide, the gene sequence of the GFP, and the DNA of the TEV protease digestion sequence to codon optimization, and inserting a resulting synthesized gene between Nhe I and Xho I digestion sites of a plasmid pcDNA3.1 to obtain the expression vector, denoted as pcDNA3.1-GFP-CAL3a1.

More preferably, the secretion signal peptide is an α-mannosidase signal peptide.

The present disclosure further provides an expression method for rhCol III a1 fusion protein, including transfecting the expression vector into a human embryonic kidney cell Expi293F to allow expression.

Preferably, a culture method of the human embryonic kidney cell Expi293F includes the following steps: subjecting the human embryonic kidney cell Expi293F to pre-suspension culture in a serum-free medium on a shaker at 37° C., and 110 rpm with a humidity of 80% under 7% carbon dioxide.

Preferably, the transfecting is conducted when a resulting cultured human embryonic kidney cell Expi293F reaches a cell density of (2.5-3.0)×10⁶ cells/mL and a cell viability of greater than 95%.

The present disclosure further provides an rhCol III a1 fusion protein expressed by the expression method, where the rhCol III a1 fusion protein has an amino acid sequence set forth in SEQ ID NO: 3.

The present disclosure further provides the use of rhCol III a1 or the rhCol III a1 fusion protein in the preparation of a product for promoting a cell migration activity.

The present disclosure further provides the use of rhCol III a1 or the rhCol III a1 fusion protein in the preparation of a product for skin repair.

Compared with the prior art, the present disclosure has the following beneficial effects:

In the present disclosure, a gene sequence on positions 154 to 1.221 of a humanized collagen α chain is selected and ligated to a vector pcDNA3.1, the secretion signal peptide and GFP are added to the N-terminus of a target protein to optimize the expression vector. A resulting optimized expression vector is transfected into a human embryonic kidney cell Expi293F, and then rhCol III a1 is secreted to form a fusion protein with a soluble GFP. The fusion protein of the GFP and rhCol III has an activity of promoting the migration of α1 BALB/c 3T3 cells. Tests have shown that a 100 μg/mL recombinant collagen experimental group (scratch recovery rate 49%) shows significant activity in promoting the cell migration compared with a control group (scratch recovery rate 14%) after 6 hours of action. After 12 hours of action, the experimental group (100 μg/mL) shows a scratch recovery rate of 96%, which is much higher than the percentage of 38% in the control group. Activity studies have shown that the GFP-fused rhCol III a1 expressed in the present disclosure has desirable activity in promoting cell migration and exhibits a huge application potential.

### BRIEF DESCRIPTION OF THE DRAWINGS

FIG. 1 shows a map of the expression vector of rhCol III a1-fused GFP:

FIG. 2 shows an SDS-PAGE electrophoresis pattern of the rhCol III a1-fused GFP:

FIG. 3 shows experimental results of the rhCol III a1-fused GFP in promoting cell migration:

FIG. 4 shows the experimental results of rhCol III a1 and the rhCol III a1-fused GFP in promoting cell migration; and

FIG. 5 shows a statistical graph of the scratch recovery rate of the rhCol III a1-fused GFP in promoting cell migration.

### DETAILED DESCRIPTION OF THE EMBODIMENTS

The present disclosure provides rhCol III a1, where rhCol III a1 has the nucleotide sequence set forth in SEQ ID NO: 1. In the present disclosure, the nucleotide sequence of rhCol III a1 is subjected to codon optimization and then synthesized by Sangon Biotech Co., Ltd.

The present disclosure further provides an expression vector of rhCol III a1, including the nucleotide sequence set forth in SEQ ID NO: 1, a gene sequence of a secretion signal peptide, a gene sequence of a GFP, and a DNA of a tobacco etch virus (TEV) protease digestion sequence.

In the present disclosure, a preparation method of the expression vector includes the following steps: subjecting the gene sequence of the secretion signal peptide, the gene sequence of the GFP, and the DNA of the TEV protease digestion sequence to codon optimization, and inserting a resulting synthesized gene between Nhe I and BamH I digestion sites of a plasmid pcDNA3.1 to obtain an expression vector, denoted as pcDNA3.1-GFP: where the gene sequence of the secretion signal peptide, the gene sequence of the GFP, and the DNA of the TEV protease digestion sequence has a synthetic gene set forth in SEQ ID NO: 2. The gene sequence SEQ ID NO: 1 of rhCol III a1 is inserted between BamH I and Xho I digestion sites of the pcDNA3.1-GFP vector to obtain the expression vector capable of expressing a fusion protein of the GFP and rhCol III a1, denoted as pcDNA3.1-GFP-CAL3a1. The expression vector pcDNA3.1-GFP is synthesized by Sangon Biotech Co., Ltd. Expression of the GFP by a GFP gene can increase the expression level of the target protein. For the DNA of the TEV protease digestion sequence, the expressed TEV protease can recognize eight amino acid sequences. After being cleaved by the TEV protease, the GFP is released to obtain a GFP-free rhCol III a1.

In the present disclosure, the secretion signal peptide is an α-mannosidase signal peptide: the secretion signal peptide is synthesized by the company from the secretion signal peptide of α-mannosidase in Trypanosoma cruzi. Fusion of the secretion signal peptide in a plasmid of the pcDNA3.1 vector can help the recombinant protein expressed by the target gene to be secreted into a cell medium, thereby increasing the expression level of the recombinant protein. The secretion signal peptide can be cleaved by a signal peptidase after completion of expression.

The present disclosure further provides an expression method for rhCol III a1 fusion protein, including transfecting the expression vector into a human embryonic kidney cell Expi293F to allow expression. The human embryonic kidney cell Expi293F is derived from Thermo: a14528. The transfecting is conducted using a plasmid transfection kit derived from Beyotime: C0518.

In the present disclosure, a culture method of the human embryonic kidney cell Expi293F includes the following steps: subjecting the human embryonic kidney cell Expi293F to pre-suspension culture in a serum-free medium on a shaker at 37° C., and 110 rpm with a humidity of 80% under 7% carbon dioxide. The serum-free medium is derived from Genetimes ExCell Bio: HE000-N012.

In the present disclosure, the transfecting is conducted when a resulting cultured human embryonic kidney cell Expi293F reaches a cell density of (2.5-3.0)×10⁶ cells/mL and a cell viability of greater than 95%.

The present disclosure further provides an rhCol III a1 fusion protein expressed by the expression method, where the rhCol III a1 fusion protein has an amino acid sequence set forth in SEQ ID NO: 3.

The present disclosure further provides the use of rhCol III a1 or the rhCol III a1 fusion protein in the preparation of a product for promoting a cell migration activity: In the present disclosure. rhCol III a1 is subjected to cell migration activity assay. Assay results have shown that a 100 μg/mL recombinant collagen experimental group (scratch recovery rate 49%) shows significant activity in promoting the cell migration compared with a control group (scratch recovery rate 14%) after 6 h of action. After 12 hours of action, the experimental group showed a scratch recovery rate of 96%, which is much higher than the percentage of 38% in the control group. The GFP-fused rhCol III a1 expressed in the present disclosure has a desirable activity in promoting cell migration and exhibits a huge application potential, and exhibits a huge application potential in the preparation of a product that promotes cell migration activity.

The present disclosure further provides the use of rhCol III a1 or the rhCol III a1 fusion protein in the preparation of a product for skin repair.

The technical solutions provided by the present disclosure will be described in detail below with reference to the examples, but they should not be construed as limiting the claimed scope of the present disclosure.

### Example 1 Constructing an Expression Vector of rhCol III a1

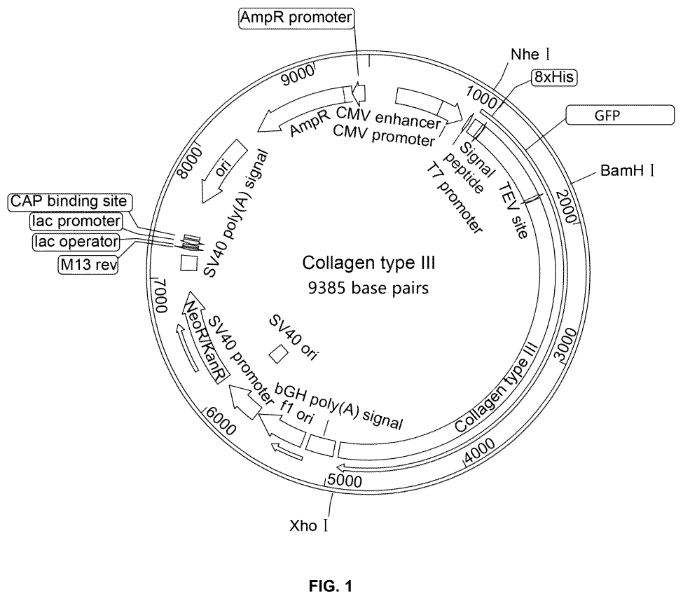

Sangon Biotech Co., Ltd, was entrusted to synthesize a gene sequence of a secretion signal peptide of α-mannosidase from Trypanosoma cruzi, a gene sequence of a GFP protein, and a DNA of a TEV protease digestion sequence, which were subjected to codon optimization, and a resulting synthesized gene was inserted between Nhe I and BamH I digestion sites of a plasmid pcDNA3.1 to obtain a GFP expression vector, denoted as pcDNA3.1-GFP. Sangon Biotech Co., Ltd, was entrusted to synthesize a DNA corresponding to an amino acid sequence (SEQ ID NO: 1) at positions 154 to 1.221 of a humanized collagen type III a1 chain, which was subjected to codon optimization, and a resulting synthesized gene was inserted between BamH I and Xho I digestion sites of the vector pcDNA3.1-GFP, and an expression vector capable of expressing a fusion protein of the GFP and rhCol III a1 was obtained, denoted as pcDNA3.1-GFP-CAL3a1 (FIG. 1).

The plasmid expression vector was transformed into E. coli DH5a, inoculated into an LB broth (each liter of the broth included 10 g peptone. 5 g yeast powder, and 10 g NaCl), incubated at 37° C. 200 rpm for 2 hours, coated on an LB solid medium plate (0.1 mg/mL ampicillin), incubated upside down in an incubator at 37° C. for 12 hours, and a single colony was selected and added into 50 mL of LB broth (0.1 mg/mL ampicillin) to allow incubation at 37° C., and 200 rpm for 12 hours. The plasmid was extracted with a plasmid bulk extraction kit (Beyotime: D0025-3), and a concentration of the expression vector was detected with a Nanodrop instrument. The expression vector could be stored at 4° C. for one month and was recommended to be used within a week.

### Example 2 Expression and Purification of rhCol III a1

The Expi293F cells were subjected to pre-suspension culture in a serum-free medium on a shaker at 37° C., and 110 rpm with a humidity of 80% under 7% carbon dioxide. The plasmid transfecting was conducted when the cell density reached (2.5-3.0)×10⁶ cells/mL and a cell viability of greater than 95%. The transfection of the Expi293F cells was conducted with a plasmid transfection kit (taking 40 mL as an example): 80 μL of transfection reagent was added into 1 mL of cell medium, and mixed gently to obtain a solution A: 40 μg of plasmid extracted in Example 1 was added into 1 mL of cell medium, and mixed gently to obtain a solution B. The solution B was gently mixed with the solution A and allowed to stand for 15 min. The mixture of A and B was added to 40 mL of Expi293F cells obtained by the suspension culture, and the culture was continued for 6 days to 7 days, cell viability was checked every day, and the cells were collected when the cell viability was not more than 50%.

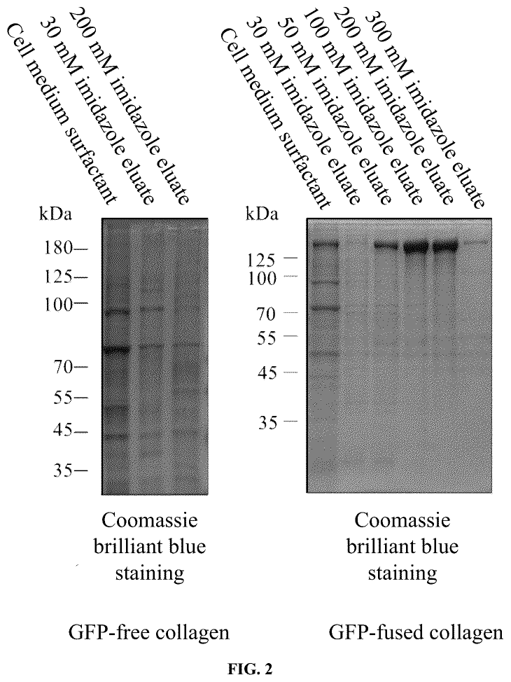

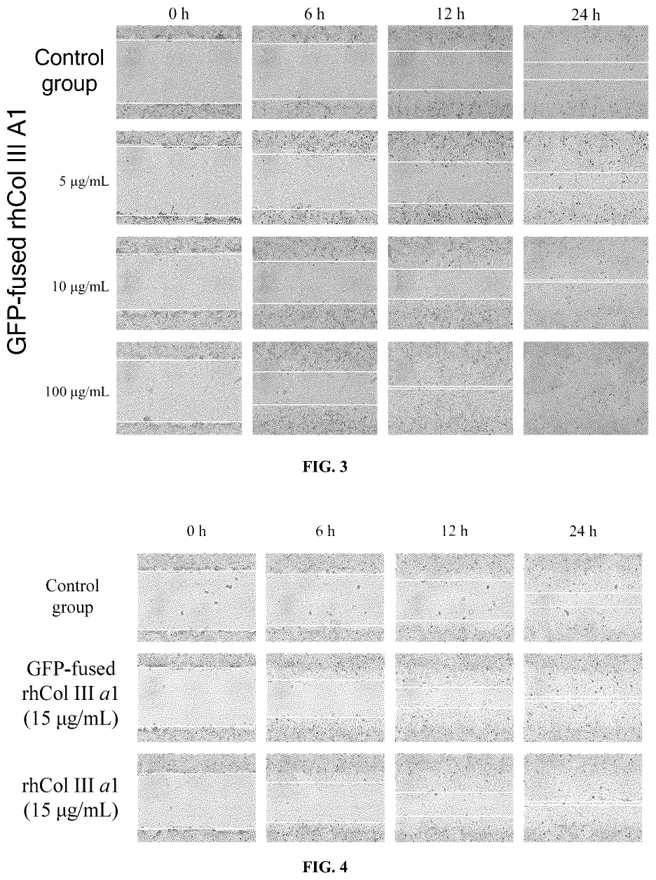

The resulting cell suspension was centrifuged at 11.000 rpm for 10 min at 4° C., an obtained supernatant was incubated with a well-balanced 5 mL nickel column (Thermo Fisher: 88221), eluted by gravity, the nickel column was washed with 50 mL of PBS solution containing 10 mM imidazole, gradient-eluted with PBS solutions containing 30 mM. 50 mM. 100 mM. 200 mM, and 300 mM imidazole separately, and the protein was detected by SDS-PAGE: obtained PBS elutions containing the target protein with high purity were combined, and the imidazole was removed by an ultrafiltration tube (Millipore. UFC9010), as shown in FIG. 2. As shown in FIG. 2, there was basically no collagen expression in the absence of GFP (left side). The GFP-fused expression vector had obvious bands when being expressed, a relatively pure fusion protein of the GFP and rhCol III a1 was obtained after purification on the nickel column. The results showed that the rhCol III a1 fusion protein obtained in the present disclosure had an expression level higher than that of the non-fusion protein.

### Example 3 Determination of rhCol III a1 in Promoting Cell Migration Activity

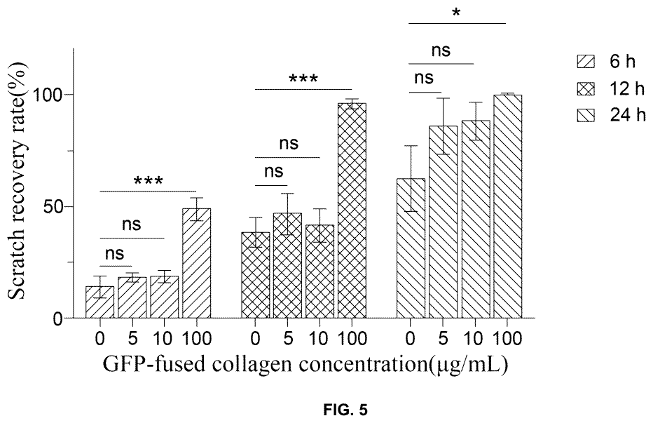

On the back of a 6-well plate, horizontal lines were evenly drawn with a marker using a ruler, where one horizontal line was drawn every 0.5 cm to 1 cm, and 5 horizontal lines were drawn for each well. About 5*105 BALB/c 3T3 cells were added to each well and cultured overnight to make the cells adhere to the wall. The next day, the cells were streaked with a sterile tip using a ruler, and washed 3 times with PBS to remove the streaked cells: a DMEM medium (containing 1-2% FBS) was added to set up a control group (only adding PBS solution) and experimental groups with three concentrations of rhCol III a1 fusion protein (purified target protein in Example 2) of 5 μg/mL, 10 μg/mL, and 100 μg/mL, and the cells were returned to the incubator. Sampling and taking photos were conducted at 0 h, 6 h, 12 h, and 24 h. Three photos were taken for each experiment, and scratch areas were analyzed and calculated using software ImageJ to obtain the scratch recovery rate at different times. Recovery rate %=(scratch area at time 0—scratch area at a corresponding time)/scratch area at time 0*100. FIG. 5 showed the data of three independent experiments (mean±standard deviation), t-test, where P<0.05 was * (statistically significant difference), P<0.01 was ** and P<0.001 was ***.

As shown in FIG. 5, a 100 μg/mL recombinant collagen experimental group (scratch recovery rate 49%) showed significant activity in promoting the cell migration compared with a control group (scratch recovery rate 14%) after 6 hours of action. After 12 hours of action, the experimental group (100 μg/mL) showed a scratch recovery rate of 96%, which was much higher than the percentage of 38% in the control group.

According to the experimental steps of this example, the control group (only adding PBS solution), 15 μg/mL rhCol III a1 fusion protein (the purified target protein in Example 2), and 15 μg/mL rhCol III a1 were set up.

The fusion protein obtained in the present disclosure had comparable activity to the non-fusion protein after the excision of GFP (FIG. 4).

The above are merely preferred implementations of the present application. It should be noted that a person of ordinary skill in the art may further make several improvements and modifications without departing from the principle of the present application, but such improvements and modifications should be deemed as falling within the protection scope of the present application.

1.-14. (canceled)

15. A recombinant humanized collagen type III alpha-1 (rhCol III a1) fusion protein, wherein the rhCol III a1 fusion protein has the amino acid sequence set forth in SEQ ID NO: 3; and wherein the rhCol III a1 fusion protein is obtained by transfecting an expression vector into a human embryonic kidney cell Expi293F to allow expression, wherein the expression vector comprises a nucleotide sequence encoding rhCol Ill a1, a gene sequence of a secretion signal peptide, a gene sequence of a green fluorescent protein (GFP), and DNA of a tobacco etch virus (TEV) protease digestion sequence;
wherein the nucleotide sequence encoding rhCol III a1 has the sequence set forth in SEQ ID NO: 1.

16. The rhCol III a1 fusion protein according to claim 15, wherein a preparation method for the expression vector comprises the following steps: subjecting the nucleotide sequence set forth in SEQ ID NO: 1, the gene sequence of the secretion signal peptide, the gene sequence of the GFP, and the DNA of the TEV protease digestion sequence to codon optimization, and inserting a resulting synthesized gene between Nhe I and Xho I digestion sites of a plasmid pcDNA3.1 to obtain the expression vector, denoted as pcDNA3.1-GFP-CAL3a1.

17. The rhCol Ill a1 fusion protein according to claim 16, wherein the secretion signal peptide is an α-mannosidase signal peptide.

18. The rhCol Ill a1 fusion protein according to claim 15, wherein a culture method for the human embryonic kidney cell Expi293F comprises the following steps: subjecting the human embryonic kidney cell Expi293F to pre-suspension culture in a serum-free medium on a shaker at 37° C., and 110 rpm with a humidity of 80% under 7% carbon dioxide.

19. The rhCol III a1 fusion protein according to claim 18, wherein the transfecting is conducted when a resulting cultured human embryonic kidney cell Expi293F reaches a cell density of (2.5-3.0)×10⁶ cells/mL and a cell viability of greater than 95%.

20. Use of the rhCol III a1 fusion protein according to claim 15 in preparation of a product for skin repair.

21. Use of the rhCol III a1 fusion protein according to claim 16 in preparation of a product for skin repair.

22. Use of the rhCol III a1 fusion protein according to claim 17 in preparation of a product for skin repair.

23. Use of the rhCol III a1 fusion protein according to claim 18 in preparation of a product for skin repair.

24. Use of the rhCol III a1 fusion protein according to claim 19 in preparation of a product for skin repair.
